(19) United States
(12) Patent Application Publication
(10) Pub. No.: US 2026/0196353 A1
(43) Pub. Date: Jul. 9, 2026

(54) SYSTEM, METHOD AND COMPUTER-ACCESSIBLE MEDIUM FOR FACILITATING CANCER SCREENING AND RISK PROFILING

(71) Applicant: NEW YORK UNIVERSITY, New York, NY (US)

(72) Inventors: Krzysztof J. GERAS, Hoboken, NJ (US); Jungkyu PARK, Yorba Linda, CA (US); Yiqiu SHEN, Jersey City, NJ (US)

(21) Appl. No.: 19/559,738

(22) Filed: Mar. 6, 2026

Related U.S. Application Data
(63) Continuation of application No. PCT/US2024/046417, filed on Sep. 12, 2024.
(60) Provisional application No. 63/537,938, filed on Sep. 12, 2023.

(51) Int. Cl. G16H 50/20; G06N 20/00; G16H 20/10; G16H 30/40; G16H 40/20; G16H 50/30; G16H 50/70

(52) U.S. Cl. CPC G16H 50/20; G06N 20/00; G16H 20/10; G16H 30/40; G16H 40/20; G16H 50/30; G16H 50/70

(57) ABSTRACT

Exemplary systems, methods and Computer-accessible medium according to the exemplary embodiments of the present disclosure can provide a Multi-modal Transformer (MMT), a neural network that synergistically utilizes mammography and ultrasound to identify existing cancers and estimate future cancer risk. MMT aggregates multi-modal data through self-attention and modeling temporal tissue changes by comparing current exams to prior imaging. Thus, exemplary method, system and computer-accessible medium can be provided for detecting cancer. with which it possible to receive, with an artificial intelligence (AI) procedure, a plurality of scanning images associated with multiple modalities for at least one portion of a body, train the AI procedure on a multi-modal image dataset based on the plurality of scanning images, and predict, by the trained AI procedure, an existence of the cancer based on the multiple modalities of the plurality of scanning images.

### CROSS REFERENCE TO RELATED APPLICATIONS

This application relates to and claims the benefit of priority from U.S. Provisional Patent Application No. 63/537,938, filed on Sep. 12, 2023, the entire disclosure of which is incorporated herein by reference.

### FIELD OF THE DISCLOSURE

The present disclosure relates generally to breast cancer screening, and more specifically, to multimodal artificial intelligence systems, methods and computer-accessible medium for detecting and predicting cancer, using, e.g., one or more multi-modal transformer(s).

### BACKGROUND INFORMATION

Breast cancer is the leading cause of cancer death in women globally. Breast cancer screening aims to detect cancer in its early stage of development so that treatment can lead to better patient outcomes. Despite the wide adoption of digital breast tomosynthesis (DBT) and full-field digital mammography (FFDM), only approximately 75% of breast cancers are diagnosed mammographically. (See, e.g., Lee et al., 2021; and Monticciolo et al., 2017). This limitation stems from dense breast tissue obscuring smaller tumors and reducing mammography's sensitivity to 61-65% in women with extremely dense breasts. (See, e.g., Mandelson et al., 2000; Wanders et al., 2017; and Destounis et al., 2017). These women require supplemental screening to compensate for the limitations of mammography. Ultrasound is commonly used given its accessibility, lower costs, and lack of radiation. While ultrasound does increase cancer detection rates by 3-4 per 1000 women (see, e.g., Berg et al., 2012), this improvement comes at the cost of lower specificity, increased recall rates of 7.5-10.6% (see, e.g., Berg and Vourtsis, 2019; Brem et al., 2015; and Butler and Hooley, 2020) and lower positive predictive values (PPV) of 9-11% (see, e g., Berg and Vourtsis, 2019), leading to unnecessary diagnostic imaging and biopsies. Artificial intelligence presents opportunities to improve precision by synergistically using mammography and ultrasound.

Deep learning models have been applied to support breast cancer screening, primarily through detecting existing cancers (see, e.g, Shen et al., 2021b; Wu et al., 2019; McKinney et al., 2020; Shen et al., 2019a; Lotter et al., 2021; Rodriguez-Ruiz et al., 2019; and Lotter et al., 2021) or predicting future risk (see, e.g., Yala et al., 2019, 2021; Arasu et al., 2023; Lehman et al., 2022). Within this area, several seminal studies have made great contributions. McKinney et al. (2020) demonstrated that convolutional neural networks (CNNs) match the screening performance of radiologists and retain generalizability across countries. Yala et al. (2019, 2021) proposed Mirai, an artificial intelligence (AI) system that utilizes mammography and clinical risk factors to fore-cast the future risk of breast cancer. Shen et al. (2021) showed that AI can reduce the false-positive rates by 37.3% in breast ultrasound interpretation, without compromising sensitivity.

Despite these advances, there are two major limitations with such prior technologies. First, existing works concentrate on a single imaging modality, missing cross-modal patterns only visible through integrating multiple imaging modalities. In contrast, radiologists often use complementary imaging modalities to ascertain a diagnosis and increase accuracy. (See, e.g., Bankman, 2008). Furthermore, existing work overlooks the utility of prior imaging. However, a comparison with two or more prior mammograms has been shown to significantly reduce the recall rate and increase cancer detection rate and PPV1. (See, e.g., Hayward et al., 2016).

Thus, it may be beneficial to provide exemplary systems, methods and computer-accessible medium can overcome at least some of the deficiencies described herein above, including, e.g., the exemplary AI systems, methods and computer-accessible medium which can be configured to reference prior imaging and synthesizing information from mammography, ultrasound and other modalities.

### SUMMARY OF THE EXEMPLARY EMBODIMENTS

Thus, AI systems, methods and computer-accessible medium according to the exemplary embodiments of the present disclosure can be provided which can have functionalities of, e.g., detecting extant cancers and predicting future cancer risk, and overcome at least some of the deficiencies described herein above.

The following is intended to be a brief summary of the exemplary embodiments of the present disclosure, and is not intended to limit the scope of the exemplary embodiments.

In some exemplary aspects, the exemplary systems, methods, and non-transitory computer accessible medium according to the present disclosure can be provided for receiving a plurality of scanning images associated with multiple modalities for a portion of a body, training the artificial intelligence procedure on a multi-modal image dataset based on the plurality of scanning images, and predicting, by the trained artificial intelligence procedure, an existence of the cancer based on the multiple modalities of the plurality of scanning images. In some exemplary embodiments, the multiple modalities can include full-field digital mammography, ultrasound, digital breast tomosynthesis, and any other suitable imaging technology.

In some exemplary aspects, the exemplary systems, methods, and non-transitory computer accessible medium according to the exemplary embodiments of the present disclosure may relate to predicting the current existence of cancer in a patient and/or predicting a likelihood that a patient will develop cancer within a future time frame, which in some embodiments may be up to five years or longer.

According to further exemplary aspects, the exemplary systems, methods, and non-transitory computer accessible medium according to the exemplary embodiments of the present disclosure may further include determining at least one suspicious region from each of the plurality of scanning images and extracting a set of feature vectors from each scanning image for the at least one suspicious region, wherein the artificial intelligence procedure is trained on the sets of feature vectors extracted from the scanning images. In some exemplary embodiments, the trained artificial intelligence procedure may aggregate the scanning images over time and the cancer prediction may be time-based.

In yet further exemplary aspects, the exemplary systems, methods, and non-transitory computer accessible medium according to the exemplary embodiments of the present disclosure may include ordering a treatment based on the prediction. Such treatment, according to the exemplary embodiments of the present disclosure, can include one or more of increased monitoring, a referral to an oncologist, and a cancer treatment.

In some exemplary aspects, the exemplary systems, methods, and non-transitory computer accessible medium according to the present disclosure can be provided for receiving, by an artificial intelligence procedure, a plurality of scanning images comprising the scan of a body part at multiple discrete points in time, predicting the existence of cancer based on the body part scans at multiple discrete points in time, and training the artificial intelligence algorithm on a time-based image dataset.

These and other objects, features and advantages of the exemplary embodiments of the present disclosure will become apparent upon reading the following detailed description of the exemplary embodiments of the present disclosure, when taken in conjunction with the appended numbered claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary illustration of a multimodal transformer architecture according to certain exemplary embodiments of the present disclosure;

FIG. 2 is an illustration of an exemplary block diagram of an exemplary system in accordance with certain exemplary embodiments of the present disclosure;

FIG. 3 is an exemplary flow chart of an exemplary method for generating a model in accordance with certain exemplary embodiments of the present disclosure; and

FIG. 4 is an illustration of an exemplary flow chart of an exemplary method for generating malignancy predictions in accordance with certain exemplary embodiments of the present disclosure.

Throughout the drawings, the same reference numerals and characters, unless otherwise stated, are used to denote features, elements, components or portions of the illustrated embodiments. Moreover, while the present disclosure will now be described in detail with reference to the figures, it is done so in connection with the illustrative embodiments and is not limited by the particular embodiments illustrated in the figures and the appended numbered claims.

### DETAILED DESCRIPTION OF EXAMPLE EMBODIMENTS

The following description of the exemplary embodiments provides non-limiting representative examples referencing numerals to particularly describe features and teachings of different aspects of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

### 1. Exemplary Methods

Exemplary Problem Formulation: According to the exemplary embodiments of the present disclosure, it is possible to provide systems, methods and computer-accessible medium to provide cancer diagnosis (e.g., breast cancer diagnosis) as a sequence classification task. For example, let Sᵢ denote an imaging exam with images

$${I}_{i}^{1} , {I}_{i}^{2} , ... , {I}_{i}^{{l}_{i}}$$

where lᵢ denotes the number of images in Sᵢ. Let mᵢ denote Sᵢ's imaging modality and tᵢ denote the time when Sᵢ is performed. Sᵢ has prior exams

$${S}_{i}^{1} , {S}_{i}^{2} , ... , {S}_{i}^{{r}_{i}} ,$$

all of which can belong to the same patient but may have been performed at the same or earlier times

$${t}_{i}^{1} , {t}_{i}^{2} , ... , {t}_{i}^{{r}_{i}} \leq {t}_{i} ,$$

where rᵢ is the number of prior exams. Exemplary embodiments of the present disclosure build an AI system that takes the sequence

$${Q}_{i} = \{ {S}_{i} , {S}_{i}^{1} , {S}_{i}^{2} , ... , {S}_{i}^{{r}_{i}} \}$$

as an input and makes a series of probabilistic predictions

$$y {\hat{}}_{i}^{j} ∈ [ 0 , 1 ]$$

quantifying the probability of malignancy within 120 days (j=0) and each of 1-5 years (j=1-5) from tᵢ.

The systems, methods and computer-accessible medium according to the exemplary embodiments of the present disclosure can work with the following important challenges. First, each patient has a unique exam history (and hence a unique set of imaging modalities) with variable numbers of images. Exemplary models according to the exemplary embodiments of the present disclosure can handle this variability. Second, malignant lesions can have diverse visual patterns across modalities. Exemplary models according to the exemplary embodiments of the present disclosure can capture this spectrum and integrate findings across imaging modalities.

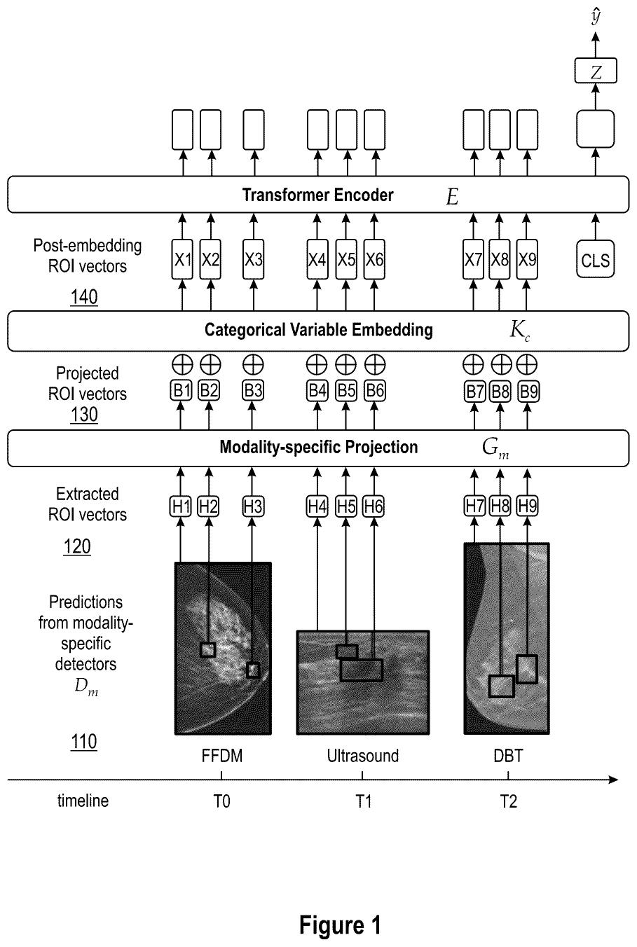

Exemplary Multi-modal Transformer: The systems, methods and computer-accessible medium according to the exemplary embodiments of the present disclosure can utilize a Multimodal Transformer (MMT) to address the aforementioned challenges. As illustrated in FIG. 1, the MMT of the exemplary embodiments of the systems, methods and computer-readable medium of the present disclosure can produce cancer predictions in the following exemplary steps/procedures. First, the MMT of the exemplary embodiments can apply modality-specific detectors 110 on all images in the sequence to extract feature vectors from regions that are suspicious of cancer 120. Second, the MMT of the exemplary embodiments can combine these exemplary features 120 with embeddings of non-image variables 130. Third, the post-embedding features 140 can be fed into a transformer encoder to detect temporal changes in tissue patterns, integrate multi-modal tissue information, and produce malignancy predictions. The following description elaborates on each such exemplary step/procedure in detail in operation and/or cooperation with the exemplary embodiments of the systems, methods and computer-accessible medium of the present disclosure.

Generating regions of interest and feature vectors: A typical input sequence Q, used by, for and/or with to the systems, methods and computer-accessible medium of an exemplary embodiment of the present disclosure can contain images of multiple modalities. Since tumor morphology can vary across modalities, the systems, methods and computer-accessible medium according to the exemplary embodiment of the present disclosure can train a detector Dₘ for each modality m ∈ {FFDM, Ultrasound, DBT}. Each Dₘ can accept an image I as input and outputs kₘ regions of interest (ROIs) with feature representations H ∈ R^{k}^{ₘ}^{,d}^{ₘ} and score P_{c} ∈ R^{k}^{ₘ}, reflecting a belief that each ROI contains a malignant lesion. In certain exemplary embodiments of the present disclosure, e.g., only the top kₘ highest scoring ROIs may be extracted, where kₘ is a hyper-parameter that can be tuned on the validation set. According to certain exemplary embodiments of the present disclosure, as feature vectors are extracted by different Dₘ, they may vary in size and scale. To address this, the the systems, methods and computer-accessible medium according to the exemplary embodiments of the present disclosure can apply a modality-specific transform Gm (a multilayer perceptron) to project all features into the same embedding space:

$$B = {G}_{m} ( H ) , \qquad ( 1 )$$

where B ∈ R^{k}^{ₘ}^{,d} denotes the post-projection feature vectors.

Categorical embeddings. The systems, methods and computer-accessible medium according to the exemplary embodiments of the present disclosure can incorporate and/or utilize categorical variables including study date, laterality, imaging modality, imaging view angles, and patient age discretized into ranges (<40, 40-50, 50-60, 60-70, >70). According to certain exemplary embodiments of the present disclosure, it is possible to utilize the embedding technique to map each variable c to an embedding vector K_{c} ∈ R¹⁰⁰, which can then be concatenated with the post-projection ROI feature vectors. In addition, it is possible to use a multilayer perceptron f_{emb}: R^{d+500} 1→R^{d} to reduce dimensionality:

$$X = {f}_{emb} ( {[ B , {K}_{date} , {K}_{lat} , {K}_{mod} , {K}_{view} , {K}_{age} ]}^{T} ) , \qquad ( 2 )$$

where X ∈ R^{k}^{ₘ}^{,d} denote the post-embedding ROI vectors.

Exemplary Transformer. The systems, methods and computer-accessible medium according to the exemplary embodiments of the present disclosure can use, e.g., a transformer encoder (Vaswani et al., 2017) to facilitate an interaction among post-embedding ROI vectors from all images. The transformer encoder can use multi-head attention to selectively combine information from the input sequence. As a common practice (see, e.g., Devlin et al., 2018), the systems, methods and computer-accessible medium according to the exemplary embodiments of the present disclosure can be used to inject a special CLS token into the input sequence. The CLS token can condense variable-length input sequences into a fixed-size aggregated representation and allow the transformer to iteratively update it using signals from all post-embedding ROI vectors:

$$CLS ’ = transformer ( [ X , CLS ] ) . \qquad ( 3 )$$

Next, the systems, methods and computer-accessible medium according to the exemplary embodiments of the present disclosure can apply a multi-layer perceptron Z: R^{d}→R⁶ with a rectified linear unit (ReLU) on the post-transformer CLS vector (CLS′) to generate six non-negative risk scores for nonoverlapping intervals: baseline risk within 120 days (L⁰), additional 120d—1 yr risk (L¹), 1-2 yr risk (L²), 2-3 yr risk (L³), 3-4 yr risk (L⁴), and 4-5 yr risk (L⁵). This is expressed in the equation below:

$${[ {L}^{0} , {L}^{1} , {L}^{2} , {L}^{3} , {L}^{4} , {L}^{5} ]}^{T} = Re LU ( Z ( CLS ’ ) ) . \qquad ( 4 )$$

Further, the systems, methods and computer-accessible medium according to the exemplary embodiments of the present disclosure can use an additive hazard layer (see, e.g., Aalen and Scheike, 2005) with sigmoid non-linearity to generate the cumulative probability of malignancy:

$${\hat{y}}^{j} = σ ( {L}^{O} + {\sum }_{k = 1}^{j} {L}_{k} ) . \qquad ( 5 )$$

Exemplary Training: The MMT according to the exemplary embodiments of the present disclosure can be trained in the following exemplary phases. First, for each modality, the exemplary systems and methods can independently train cancer detectors using only images from that modality. FFDM and DBT detectors can be parameterized as YOLOX (Ge et al., 2021), MogaNet (see, e.g., Li et al., 2022), and GMIC (see, e.g., Shen et al., 2021a, 2019b). YOLOX is an anchor-free version of YOLO (see, e.g., Redmon et al., 2016), a popular object detection model family. MogaNet is a CNN that can efficiently model interactions among visual features. GMIC is a resource-efficient CNN that is designed for high-resolution medical images. For the DBT, the exemplary embodiments of the present disclosure can be trained on 2D slices to limit computation. YOLOX and MogaNet detectors can be trained on both image and bounding box labels. To train with image labels, the exemplary systems and methods can attention-pool the features of the highest-scoring boxes and classify them using a logistic regression layer. Exemplary embodiments may use the UltraNet proposed in Shen et al. (2021a) as an ultrasound detector. For all detectors, the exemplary embodiments can extract kₘ=10 ROIs from each image.

In the second exemplary phase, the systems, methods and computer-accessible medium according to the exemplary embodiments of the present disclosure can freeze detectors and train the transformer encoder, MLPs, and embeddings on multimodal sequences using binary cross-entropy loss and Adam optimizer (see, e.g., Kingma and Ba, 2014) with a learning rate set to 10⁻⁵.

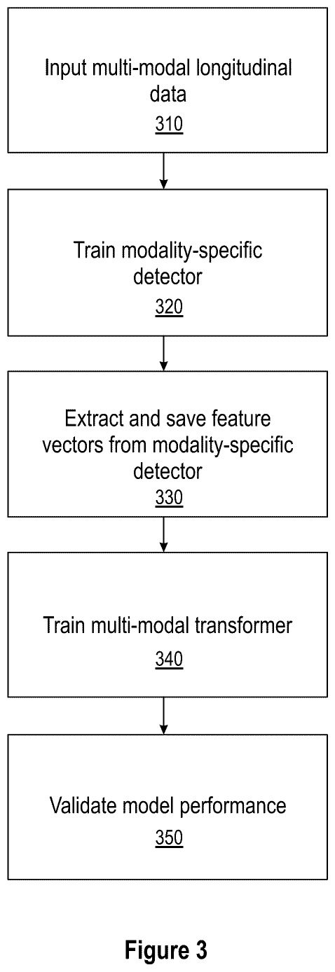

FIG. 3 illustrates a flow chart of an exemplary method for generating and training a model according to the exemplary embodiments of the present disclosure. For example, in step 310, multi-modal longitudinal data can be input. This can include receiving mammography, the DBT, and ultrasound images from current and prior exams across time. In step 320, a modality-specific detector can be trained. This can include independently training cancer detectors on images from each modality, using bounding-box annotation labels from physicians and/or pathology-driven breast-level cancer labels. In step 330, feature vectors from modality-specific detectors can be extracted and saved. This can include extracting regions of interest (ROIs) from each modality (e.g., FFDM, DBT, Ultrasound) and the associated feature vectors from each image using the trained modality-specific detectors. In step 340, the multi-modal transformer can be trained. The transformer encoder and MLPs can be trained by using the saved feature vectors from multi-modal sequences as well as non-image variables like study date, laterality, imaging modality, view angles, and patient age. Further, in step 350, the generated/trained model performance can be validated. For example, the exemplary model can be validated on a separate dataset to ensure accuracy in cancer detection and risk prediction.

Exemplary Ensembling: To improve the exemplary results, the systems, methods and computer-accessible medium according to the exemplary embodiments of the present disclosure can use model ensembling (see, e.g., Dietterich, 2000). The systems, methods and computer-accessible medium according to the exemplary embodiments of the present disclosure can train numerous MMT models, e.g., 100 MMT models, randomly combining one (1) model from each detector family with a transformer encoder that is randomly parameterized as either a DeiT (see, e.g., Touvron et al., 2021), a ViT (see, e.g., Dosovitskiy et al., 2020), or a BERT (see, e.g., Devlin et al., 2018). According to the exemplary embodiments of the present disclosure, the top 5 MMT models by validation performance can be ensemble averaged to produce the final prediction.

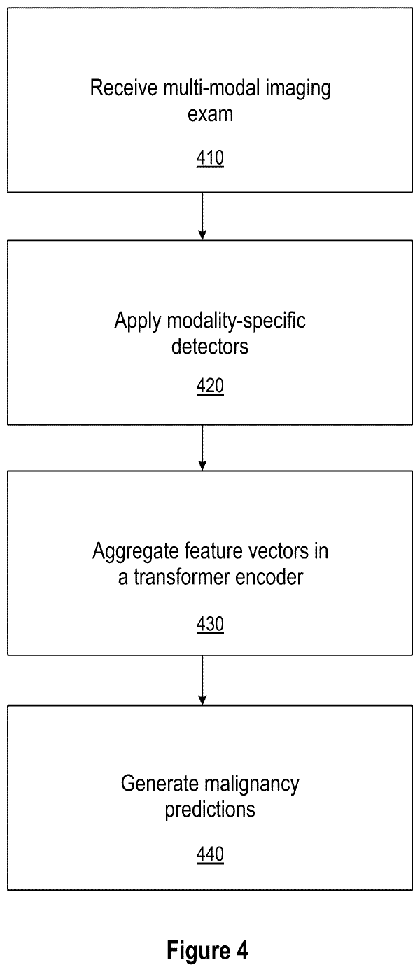

FIG. 4 shows a flow chart of an exemplary method for generating malignancy predictions using a trained model according to the exemplary embodiments of the present disclosure. In step 410, multi-modal imaging for one or more exams may be received. For example, current mammography and ultrasound images may be collected at this step, along with prior imaging if available. Then, in step 420 modality-specific detectors can be applied to the collected images. This can include the use of trained detectors to predict and extract suspicious ROIs from the images of each modality. Next, in step 430, feature vectors can be aggregated in a transformer encoder. For example, feature vectors for the ROIs, along with non-image variables, can be passed through the transformer to detect temporal changes and integrate multi-modal data. Further, malignancy predictions can be generated in step 440. For example, the exemplary model utilizing the exemplary method of FIG. 4 can produce immediate cancer predictions and long-term risk scores using the final MLP layer.

### 2. Exemplary Results

Exemplary Dataset: The systems, methods and computer-accessible medium according to the exemplary embodiments of the present disclosure can train and evaluate the model(s) on an anonymized dataset containing, e.g., 1,353,521 FFDM/DBT/ultrasound exams from, e.g., 297,751 patients who visited the anonymized institution between 2010 and 2020. Exams can be split into training (approx. 87.1%), validation (approx. 3.9%), and test (approx. 8.9%) sets, with each patient's exams assigned to only one set. Labels indicating presence or absence of cancer can be derived from corresponding pathology reports. Validation and test sets can be filtered so cancer-positive exams have pathology confirmation and cancer-negative exams have a negative follow-up. See Table AI for dataset details.

Exemplary Evaluation: The systems, methods and computer-accessible medium according to the exemplary embodiments of the present disclosure can evaluate the exemplary model's ability to detect existing cancers and predict future risks in the general screening population. Each test case is a screening visit with the required FFDM and the optional DBT/ultrasound. The exemplary model according to the exemplary embodiments of the present disclosure can utilize all available modalities and prior studies. For an existing cancer detection, a visit may be cancer-positive if a pathology study within 120 days of imaging confirms cancer. For example, the 121,037 exams in a test set, according to an exemplary embodiment, resulted in 54,789 visits with 483 positive visits. For 5-year risk stratification, the exemplary embodiments may exclude screening-detected cancers and negative cases with <5-year follow-up, focusing solely on long-term prediction. This gives 6,173 visits with 598 positive cases in the test set. Exemplary embodiments of the present disclosure can use area under the ROC curve (AUROC) and area under the precision recall curve (AUPRC) as evaluation metrics.

EXEMPLARY TABLE 1
Exemplary Cancer diagnosis performance.

| | AUROC | AUPRC |
|---|---|---|
| GMIC (Shen et al., 2021b) | 0.866 | 0.167 |
| YOLOX (Ge et al., 2021) | 0.876 | 0.172 |
| MogaNet (Li et al., 2022) | 0.874 | 0.181 |
| Multi-modal Ensemble | 0.925 | 0.251 |
| MMT | 0.943 | 0.518 |

Exemplary Performance: For cancer diagnosis, the systems, methods and computer-accessible medium according to the exemplary embodiments of the present disclosure can compare MMT to four baselines—GMIC, YOLOX, and MogaNet using FFDM only, and a multi-modal ensemble averaging predictions from the mammogram baselines and UltraNet processing ultrasound when available. The systems, methods and computer-accessible medium according to the exemplary embodiments of the present disclosure can yield the exemplary performance in Table 1. The MMT of the exemplary embodiments can achieve higher AUROC and AUPRC than mammogram-only baselines, indicating that incorporating ultrasound improves diagnostic accuracy. MMT of the exemplary embodiments can also outperform the multi-modal ensemble, indicating the transformer of the exemplary embodiments can integrate multimodal information better than simple averaging. By leveraging multiple modalities and effectively combining them via self-attention, the MMT according to the exemplary embodiments of the present disclosure can improve breast cancer diagnosis compared to both single-modality and naively combined multi-modal models.

For risk stratification, it is possible to compare the MMT of the exemplary embodiments of the present disclosure to two baselines: radiologists' BI-RADS diagnosis and Mirai Yala et al. (2019, 2021), an AI system predicting future breast cancer risk using both categorical risk factors and mammograms, on the same test set. The exemplary embodiments of the present disclosure reported the exemplary performance in Table 2. For 5-year cancer prediction, e.g., the MMT of the exemplary embodiments can achieve an AUROC of 0.826 and AUPRC of 0.524, outperforming both methods. By leveraging multimodal imaging and longitudinal patient history, MMT of the exemplary embodiments demonstrates a strong ability to predict future breast cancer risk.

EXEMPLARY TABLE 2
Exemplary Risk stratification performance.

| | AUROC | AUPRC |
|---|---|---|
| BI-RADS | 0.585 | 0.118 |
| Mirai (Yala et al., 2019) | 0.732 | 0.252 |
| MMT | 0.826 | 0.524 |

Exemplary Ablation Study: The systems, methods and computer-accessible medium according to the exemplary embodiments of the present disclosure can perform an ablation study to understand the impact of supplemental modalities and prior imaging. Exemplary embodiments can evaluate MMT according to the exemplary embodiments of the present disclosure using only mammograms with prior imaging (mammo only), using both mammograms and ultrasound but with no prior imaging (no prior), and incorporating only 1, 2, or 3 years of prior imaging.

EXEMPLARY TABLE 3
Exemplary ablation study on the impact of
supplemental modality and prior imaging.

| | |
|---|---|
| Cancer Diagnosis | Risk Stratification |

| | AUROC | AUPRC | AURPC | AUPRC |
|---|---|---|---|---|
| mammo only | 0.925 | 0.373 | 0.814 | 0.516 |
| no prior | 0.944 | 0.484 | 0.817 | 0.513 |
| 1 year prior | 0.944 | 0.487 | 0.816 | 0.514 |
| 2 year prior | 0.945 | 0.507 | 0.822 | 0.521 |
| 3 year prior | 0.944 | 0.512 | 0.825 | 0.523 |
| full model | 0.943 | 0.518 | 0.826 | 0.524 |

As shown in Table 3, compared to mammograms alone, MMT of the exemplary embodiments of the present disclosure can indicate a meaningful performance gains with ultrasound for both tasks, confirming the importance of supplemental modality. In contrast, prior imaging mainly can contribute to long term risk stratification. Moreover, prior imaging beyond two years can provide only marginal improvement. This observation is consistent with the clinical practice of using up to two years as references. Overall, the ablation highlights the value of multimodal and longitudinal information, with ultrasound and recent prior imaging improving cancer diagnosis and risk prediction.

### 3. Exemplary Discussion And Conclusion

In medical imaging, each modality can have its own strengths and limitations. Therefore, radiologists often combine multiple modalities to inform decision making. In this spirit, the exemplary embodiments of the present disclosure use MMT to jointly leverage mammography and ultrasound for breast cancer screening. Trained on a large dataset, MMT of the exemplary embodiments of the present disclosure can achieve strong performance in identifying existing cancers and predicting long-term risk.

Standard-of-care risk models generally use family history, genetic mutations, and breast density to estimate risk, but exhibit suboptimal accuracy (see, e.g., Arasu et al., 2023). This stems from their reliance on simple modeling and coarse clinical variables that inadequately capture underlying breast tissue heterogeneity associated with cancer risk. Patients with similar profiles can have very different risks. The systems, methods and computer-accessible medium according to the exemplary embodiments of the present disclosure can demonstrate that integrating multi-modal longitudinal patient data with neural networks can significantly improve risk modeling by extracting richer tissue feature representations predictive of cancer development.

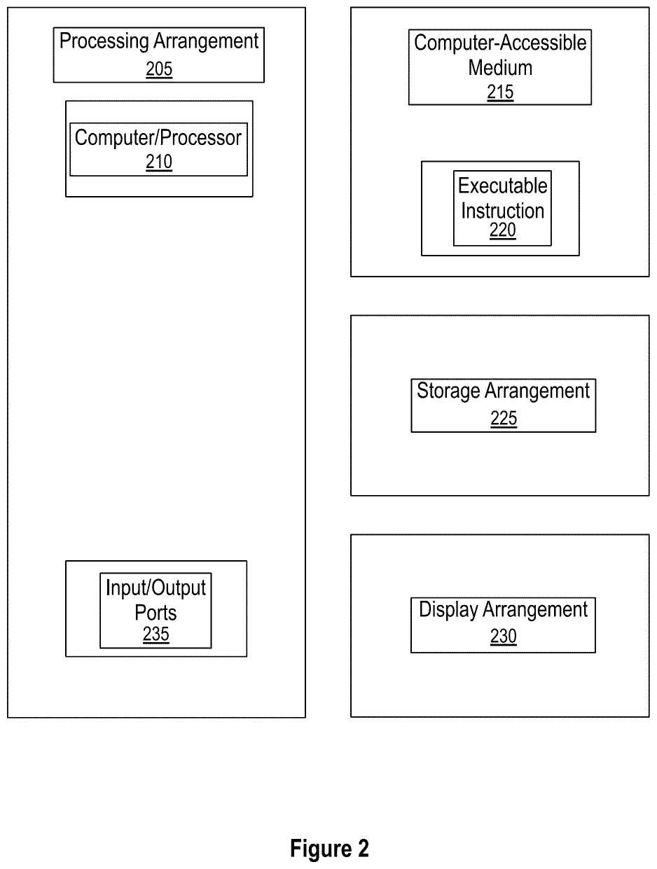

FIG. 2 shows a block diagram of an exemplary embodiment of a system according to the present disclosure. For example, the exemplary procedures in accordance with the present disclosure described herein can be performed by a processing arrangement and/or a computing arrangement (e.g., computer hardware arrangement) 205. Such processing/computing arrangement 205 can be, for example entirely or a part of, or include, but not limited to, a computer/processor 210 that can include, for example one or more microprocessors, and use instructions stored on a computer-accessible medium (e.g., RAM, ROM, hard drive, or other storage device).

As shown in FIG. 2, for example a computer-accessible medium 215 (e.g., as described herein above, a storage device such as a hard disk, floppy disk, memory stick, CD-ROM, RAM, ROM, etc., or a collection thereof) can be provided (e.g., in communication with the processing arrangement 205). The computer-accessible medium 215 can contain executable instructions 220 thereon. In addition or alternatively, a storage arrangement 225 can be provided separately from the computer-accessible medium 215, which can provide the instructions to the processing arrangement 205 so as to configure the processing arrangement to execute certain exemplary procedures, processes, and methods, as described herein above, for example. Further, the exemplary processing arrangement 205 can be provided with or include an input/output ports 235, which can include, for example a wired network, a wireless network, the internet, an intranet, a data collection probe, a sensor, etc. As shown in FIG. 2, the exemplary processing arrangement 205 can be in communication with an exemplary display arrangement 230, which, according to certain exemplary embodiments of the present disclosure, can be a touch-screen configured for inputting information to the processing arrangement in addition to outputting information from the processing arrangement, for example. Further, the exemplary display arrangement 230 and/or a storage arrangement 225 can be used to display and/or store data in a user-accessible format and/or user-readable format.

According to the exemplary embodiments of the present disclosure, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, the exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the numbered claims, and may include other examples that occur to those skilled in the art. Such other examples are intended to be within the scope of the numbered claims if they have structural elements that do not differ from the literal language of the numbered claims, or if they include equivalent structural elements with insubstantial differences from the literal language of the numbered claims.

EXEMPLARY TABLE A1
Exemplary Characteristics of the anonymized
Breast Cancer Diagnosis Multimodal Dataset.

| | Training | | Validation | | Test | |
|---|---|---|---|---|---|---|
| Patients | 263,573 | | 10,839 | | 23,339 | |
| Age, mean years (SD) | 56.98 | (13.09) | 59.58 | (11.55) | 59.77 | (11.47) |
| <40 yr old | 17,186 | (6.52%) | 173 | (1.60%) | 351 | (1.50%) |
| 40-49 yr old | 63,158 | (23.96%) | 2,308 | (21.29%) | 4,806 | (20.59%) |
| 50-59 yr old | 64,882 | (24.62%) | 3,088 | (28.49%) | 6,679 | (28.62%) |
| 60-69 yr old | 54,980 | (20.86%) | 2,960 | (27.31%) | 6,582 | (28.20%) |
| ≥70 yr old | 43,022 | (16.32%) | 2,092 | (19.30%) | 4,582 | (19.63%) |
| Unknown | 20,345 | (7.72%) | 218 | (2.01%) | 339 | (1.45%) |
| Breast density | | | | | | |
| A | 18,616 | (7.06%) | 773 | (7.13%) | 1,756 | (7.52%) |
| B | 93,990 | (35.66%) | 4,225 | (38.98%) | 9,415 | (40.34%) |
| C | 113,631 | (43.11%) | 5,005 | (46.18%) | 10,542 | (45.17%) |
| D | 16,991 | (6.45%) | 618 | (5.70%) | 1,287 | (5.51%) |
| Unknown | 20,345 | (7.72%) | 218 | (2.01%) | 339 | (1.45%) |
| Exams | 1,179,171 | | 53,313 | | 121,037 | |
| cancer 120 days | 5,586 | (1.32%) | 877 | (1.65%) | 1,902 | (1.57%) |
| cancer 120 days-1 year | 1,566 | (0.13%) | 19 | (0.04%) | 13 | (0.01%) |
| cancer 1-2 years | 4,816 | (0.41%) | 54 | (0.10%) | 133 | (0.11%) |
| cancer 2-3 years | 3,417 | (0.29%) | 249 | (0.47%) | 613 | (0.51%) |
| cancer 3-4 years | 2,089 | (0.18%) | 182 | (0.34%) | 364 | (0.30%) |
| cancer 4-5 years | 1,281 | (0.11%) | 101 | (0.19%) | 248 | (0.20%) |
| cancer >5 years | 1,391 | (0.12%) | 92 | (0.17%) | 268 | (0.22%) |
| Imaging Modality | | | | | | |
| FFDM | 546,862 | (46.38%) | 26,687 | (50.06%) | 62,249 | (51.43%) |
| DBT | 300,277 | (25.47%) | 12,139 | (22.77%) | 28,339 | (23.41%) |
| Ultrasound | 332,032 | (28.16% | 14,487 | (27.17%) | 30,449 | (25.16) |
| Exam-level BI-RADS | | | | | | |
| 0 | 439,112 | (10.99%) | 19,811 | (9.66%) | 45,060 | (9.57%) |
| 1 | 439,112 | (37.24%) | 19,811 | (37.16%) | 45,060 | (37.23%) |
| 2 | 501,915 | (42.57%) | 24,598 | (46.14%) | 56,810 | (46.94%) |
| 3 | 70,935 | (6.02%) | 1,683 | (3.16%) | 3,453 | (2.85%) |
| 4 | 33,517 | (2.84%) | 1,999 | (3.75%) | 4,002 | (3.31%)′ |
| 5 | 2,535 | (0.21%) | 73 | (0.14%) | 129 | (0.11%) |
| 6 | 1,611 | (0.14%) | 0 | (0.00%) | 0 | (0.00%) |

### EXEMPLARY REFERENCES

The following references are hereby incorporated by reference, in their entireties:
1. Odd O Aalen and Thomas H Scheike. Aalen's additive regression model. Encyclopedia of biostatistics, 1, 2005.
2. Vignesh A Arasu, Laurel A Habel, Ninah S Achacoso, Diana S M Buist, Jason B Cord, Laura J Esserman, Nola M Hylton, M Maria Glymour, John Kornak, Lawrence H Kushi, et al. Comparison of mammography ai algorithms with a clinical risk model for 5-year breast cancer risk prediction: An observational study. Radiology, 307(5):e222733, 2023.
3. Isaac Bankman. Handbook of medical image process-ing and analysis. Elsevier, 2008.
4. Wendie A Berg and Athina Vourtsis. Screening breast ultrasound using handheld or automated technique in women with dense breasts. Journal of Breast Imaging, 1(4):283-296, 2019.
5. Wendie A Berg, Zheng Zhang, Daniel Lehrer, Roberta A Jong, Etta D Pisano, Richard G Barr, Marcela B*ohm-V'elez, Mary C Mahoney, W Phil Evans, Linda H Larsen, et al. Detection of breast cancer with addition of annual screening ultra-sound or a single screening mri to mammography in women with elevated breast cancer risk. Jama, 307(13):1394-1404, 2012.
6. Rachel F Brem, Megan J Lenihan, Jennifer Lieber-man, and Jessica Torrente. Screening breast ultra-sound: past, present, and future. American Journal of Roentgenology, 204(2):234-240, 2015.
7. Reni S Butler and Regina J Hooley. Screening breast ultrasound: update after 10 years of breast density notification laws. American Journal of Roentgenology, 214(6):1424-1435, 2020.
8. Stamatia Destounis, Lisa Johnston, Ralph Highnam, Andrea Arieno, Renee Morgan, and Ariane Chan. Using volumetric breast density to quantify the potential masking risk of mammographic density. AJR Am J Roentgenol, 208(1):222-227, 2017.
9. Jacob Devlin, Ming-Wei Chang, Kenton Lee, and Kristina Toutanova. Bert: Pre-training of deep bidirectional transformers for language under-standing. arXiv preprint arXiv:1810.04805, 2018.
10. Thomas G Dietterich. Ensemble methods in machine learning. In International workshop on multiple classifier systems, pages 1-15. Springer, 2000.
11. Alexey Dosovitskiy, Lucas Beyer, Alexander Kolesnikov, Dirk Weissenborn, Xiaohua Zhai, Thomas Unterthiner, Mostafa Dehghani, Matthias Minderer, Georg Heigold, Sylvain Gelly, et al. An image is worth 16×16 words: Transformers for image recognition at scale. arXiv preprint arXiv:2010.11929, 2020.
12. Zheng Ge, Songtao Liu, Feng Wang, Zeming Li, and Jian Sun. Yolox: Exceeding yolo series in 2021. arXiv preprint arXiv:2107.08430, 2021.
13. Jessica H Hayward, Kimberly M Ray, Dorota J Wis-ner, John Kornak, Weiwen Lin, Edward A Sickles, and Bonnie N Joe. Improving screening mammography outcomes through comparison with multi-ple prior mammograms. AJR. American journal of roentgenology, 207(4):918, 2016.
14. Diederik P Kingma and Jimmy Ba. Adam: A method for stochastic optimization. arXiv preprint arXiv:1412.6980, 2014.
15. Cindy S Lee, Linda Moy, Danny Hughes, Dan Golden, Mythreyi Bhargavan-Chatfield, Jennifer Hemingway, Agnieszka Geras, Richard Duszak, and Andrew B Rosenkrantz. Radiologist characteristics associated with interpretive performance of screening mammography: a national mammography database (nmd) study. Radiology, 300(3): 518-528, 2021.
16. Constance D Lehman, Sarah Mercaldo, Leslie R Lamb, Tari A King, Leif W Ellisen, Michelle Specht, and Rulla M Tamimi. Deep learning vs traditional breast cancer risk models to support risk-based mammography screening. Journal Of The National Cancer Institute, 114(10):1355-1363, 2022.
17. Siyuan Li, Zedong Wang, Zicheng Liu, Cheng Tan, Haitao Lin, Di Wu, Zhiyuan Chen, Jiangbin Zheng, and Stan Z Li. Efficient multi-order gated aggregation network. arXiv preprint arXiv:2211.03295, 2022.
18. William Lotter, Abdul Rahman Diab, Bryan Haslam, Jiye G Kim, Giorgia Grisot, Eric Wu, Kevin Wu, Jorge Onieva Onieva, Yun Boyer, Jerrold L Boxerman, et al. Robust breast cancer detection in mammography and digital breast tomosynthesis using an annotation-efficient deep learning approach. Nature Medicine, 27(2):244-249, 2021.
19. Margaret T Mandelson, Nina Oestreicher, Peggy L Porter, Donna White, Charles A Finder, Stephen H Taplin, and Emily White. Breast density as a predictor of mammographic detection: comparison of interval- and screen-detected cancers. Journal of the National Cancer Institute, 92(13):1081-1087, 2000.
20. Scott Mayer McKinney, Marcin Sieniek, Varun Godbole, Jonathan Godwin, Natasha Antropova, Hutan Ashrafian, Trevor Back, Mary Chesus, Greg S Corrado, Ara Darzi, et al. International evaluation of an ai system for breast cancer screening. Nature, 577(7788):89-94, 2020.
21. Debra L Monticciolo, Mary S Newell, R Edward Hendrick, Mark A Helvie, Linda Moy, Barbara Mon-sees, Daniel B Kopans, Peter R Eby, and Edward A Sickles. Breast cancer screening for average-risk women: recommendations from the acrcommission on breast imaging. Journal of the American College of Radiology, 14(9):1137-1143, 2017.
22. Joseph Redmon, Santosh Divvala, Ross Girshick, and Ali Farhadi. You only look once: Unified, real-time object detection. In Proceedings of the IEEE conference on computer vision and pattern recognition, pages 779-788, 2016.
23. Alejandro Rodriguez-Ruiz, Kristina Lang, Albert Gubern-Merida, Mireille Broeders, Gisella Gennaro, Paola Clauser, Thomas H. Helbich, Margarita Chevalier, Tao Tan, Thomas Mertelmeier, et al. Stand-alone artificial intelligence for breast cancer detection in mammography: comparison with 101 radiologists. JNCI: Journal of the National Cancer Institute, 111(9):916-922, 2019.
24. Li Shen, Laurie R Margolies, Joseph H Rothstein, Eugene Fluder, Russell McBride, and Weiva Sieh. Deep learning to improve breast cancer detection on screening mammography. Scientific reports, 9 (1):12495, 2019a.
25. Yiqiu Shen, Nan Wu, Jason Phang, Jungkyu Park, Gene Kim, Linda Moy, Kyunghyun Cho, and Krzysztof J Geras. Globally-aware multiple in-stance classifier for breast cancer screening. In Ma-chine Learning in Medical Imaging: 10th International Workshop, MLMI 2019, Held in Conjunction with MICCAI 2019, Shenzhen, China, Oct. 13, 2019, Proceedings 10, pages 18-26. Springer, 2019b.
26. Yiqiu Shen, Farah E Shamout, Jamie R Oliver, Jan Witowski, Kawshik Kannan, Jungkyu Park, Nan Wu, Connor Huddleston, Stacey Wolfson, Alexandra Millet, et al. Artificial intelligence system re-duces false-positive findings in the interpretation of breast ultrasound exams. Nature communications, 12(1):5645, 2021a.
27. Yiqiu Shen, Nan Wu, Jason Phang, Jungkyu Park, Kangning Liu, Sudarshini Tyagi, Laura Heacock, S Gene Kim, Linda Moy, Kyunghyun Cho, et al. An interpretable classifier for high-resolution breast cancer screening images utilizing weakly supervised localization. Medical image analysis, 68: 101908, 2021b.
28. Hugo Touvron, Matthieu Cord, Matthijs Douze, Francisco Massa, Alexandre Sablayrolles, and Herv'e J'egou. Training data-efficient image trans-formers & distillation through attention. In International conference on machine learning, pages 10347-10357. PMLR, 2021.
29. Ashish Vaswani, Noam Shazeer, Niki Parmar, Jakob Uszkoreit, Llion Jones, Aidan N Gomez, L-ukasz Kaiser, and Illia Polosukhin. Attention is all you need. Advances in neural information processing systems, 30, 2017.
30. Johanna O P Wanders, Katharina Holland, Wouter B Veldhuis, Ritse M Mann, Ruud M Pijnappel, Petra H M Peeters, Carla H van Gils, and Nico Karssemeijer. Volumetric breast density affects performance of digital screening mammography. Breast cancer research and treatment, 162:95-103, 2017.
31. Nan Wu, Jason Phang, Jungkyu Park, Yiqiu Shen, Zhe Huang, Masha Zorin, Stanislaw Jastrzebski, Thibault Fevry, Joe Katsnelson, Eric Kim, et al. Deep neural networks improve radiologists' performance in breast cancer screening. IEEE transactions on medical imaging, 39(4):1184-1194, 2019.
32. Adam Yala, Constance Lehman, Tal Schuster, Tally Portnoi, and Regina Barzilay. A deep learning mammography-based model for improved breast cancer risk prediction. Radiology, 292(1):60-66, 2019.
33. Adam Yala, Peter G Mikhael, Fredrik Strand, Gi-gin Lin, Kevin Smith, Yung-Liang Wan, Leslie Lamb, Kevin Hughes, Constance Lehman, and Regina Barzilay. Toward robust mammography-based models for breast cancer risk. Science Translational Medicine, 13(578):eaba4373, 2021.

1. A method for detecting cancer, comprising:
receiving, with an artificial intelligence (AI) procedure, a plurality of scanning images associated with multiple modalities for at least one portion of a body;
training the AI procedure on a multi-modal image dataset based on the plurality of scanning images; and
predicting, by the trained AI procedure, an existence of the cancer based on the multiple modalities of the plurality of scanning images.

2. The method of claim 1, wherein the cancer prediction comprises a current existence of the cancer in the body.

3. The method of claim 1, wherein the cancer prediction comprises a determination of a likelihood that the body can develop the cancer within a future time frame.

4. The method of claim 3, wherein the future time frame is within five years.

5. The method of claim 1, wherein the multiple modalities comprise at least one of full-field digital mammography, ultrasound, or digital breast tomosynthesis.

6. The method of claim 1, further comprising ordering of a treatment by a physician based on the prediction.

7. The method of claim 6, wherein the treatment comprises an increased monitoring.

8. The method of claim 6, wherein the treatment comprises a referral to an oncologist.

9. The method of claim 6, wherein the treatment comprises a cancer treatment.

10. The method of claim 1, further comprising:
determining at least one suspicious region from each of the plurality of scanning images; and
extracting a set of feature vectors from each of the scanning images for the at least one suspicious region,
wherein the AI procedure is trained on the sets of feature vectors extracted from the scanning images.

11. The method of claim 1, wherein the trained AI procedure aggregates the scanning images over time, and wherein the cancer prediction is time-based.

12. A system for detecting cancer, comprising:
at least one computer processor arrangement which implements an artificial intelligence (AI) procedure configured to:
receive a plurality of scanning images comprising multiple modalities for at least one portion of a body;
be trained using a multi-modal image dataset based on the plurality of scanning images; and
predict the existence of cancer based on the multiple modalities associated with the plurality of scanning images.

13-22. (canceled)

23. A non-transitory computer-accessible medium having stored thereon computer-executable instructions for detecting cancer, which when executed by a computer arrangement, configure the computer arrangement to perform procedures comprising:
receiving, with an artificial intelligence (AI) procedure, a plurality of scanning images associated with multiple modalities for at least one portion of a body;
training the AI procedure on a multi-modal image dataset based on the plurality of scanning images; and
predicting, by the trained AI procedure, an existence of the cancer based on the multiple modalities of the plurality of scanning images.

24-33. (canceled)

34. A method for detecting cancer, comprising:
receiving, with an artificial intelligence (AI) procedure, a plurality of scanning images comprising a plurality of body part scans at multiple discrete points in time;
training the AI procedure on a time-based image dataset based on the plurality of scanning images; and
predicting, utilizing the trained AI procedure, an existence of the cancer based on the body part scan at the multiple discrete points in time.

35. A system for detecting cancer, comprising:
at least one computer processor arrangement which implements an artificial intelligence (AI) procedure configured to:
receive a plurality of scanning images comprising a plurality of body part scans at multiple discrete points in time;
be trained using a time-based image dataset based on the plurality of scanning images; and
predict the existence of cancer based on the body part scan at the multiple discrete points in time.

36. A non-transitory computer-accessible medium having stored thereon computer-executable instructions for detecting cancer, which when executed by a computer arrangement, configure the computer arrangement to perform procedures comprising:
receiving, utilizing an artificial intelligence (AI) procedure, a plurality of scanning images comprising a plurality of body part scans at multiple discrete points in time;
training the AI procedure on a time-based image dataset based on the plurality of scanning images; and
predicting, utilizing the trained AI procedure, an existence of the cancer based on the body part scan at the multiple discrete points in time.
